SEQUENCE LISTING (7 sequences)

SEQ ID NO: 1 - RNA, 20 nt, synthetic construct
  source 1..20: mol_type = other RNA
accaagtcatatgtcattaa

SEQ ID NO: 2 - DNA, 42 bp, synthetic construct
  source 1..42: mol_type = other DNA
gccctagtctagggcgcgcccaattttaataacaatttggcg

SEQ ID NO: 3 - DNA, 49 bp, synthetic construct
  source 1..49: mol_type = other DNA
caagtaaaataattggtctttaagtgacatatgacttggtagtttactg

SEQ ID NO: 4 - DNA, 63 bp, synthetic construct
  source 1..63: mol_type = other DNA
agcggccgcaaaaaaaataaaaaaaaaaaaaaaaaaaaaaaaaaaaaaatatatatattatac

SEQ ID NO: 5 - DNA, 43 bp, synthetic construct
  source 1..43: mol_type = other DNA
ttttttacaaaatgcttaagaactagcgtatgtgataacttag

SEQ ID NO: 6 - DNA, 40 bp, synthetic construct
  source 1..40: mol_type = other DNA
tattttacttggacaaattaggtaccggaagtggttctgg

SEQ ID NO: 7 - DNA, 36 bp, synthetic construct
  source 1..36: mol_type = other DNA
tttattttttttgcggccgcgcggccgcgtcccctc

(19) United States
(12) Patent Application Publication
(10) Pub. No.: US 2026/0130918 A1
(43) Pub. Date: May 14, 2026

(54) TRANSCRIPTION FACTOR PFAP2-O5 INHIBITOR FOR PLASMODIUM FALCIPARUM, PHARMACEUTICAL COMPOSITION, AND DRUG FOR TREATING MALARIA RESISTANT TO ARTEMISININ AND ANALOG THEREOF

(71) Applicants: TONGJI UNIVERSITY, Shanghai (CN); JIANGSU INSITITUTE OF PARASITIC DISEASES, Jiangsu (CN); CHINA PHARMACEUTICAL UNIVERSITY, Jiangsu (CN)

(72) Inventors: Qingfeng ZHANG, Shanghai (CN); Jun CAO, Jiangsu (CN); Xinyu YU, Jiangsu (CN); Yong YANG, Jiangsu (CN)

(73) Assignees: TONGJI UNIVERSITY, Shanghai (CN); JIANGSU INSITITUTE OF PARASITIC DISEASES, Jiangsu (CN); CHINA PHARMACEUTICAL UNIVERSITY, Jiangsu (CN)

(21) Appl. No.: 19/433,015

(22) Filed: Dec. 25, 2025

Related U.S. Application Data
(63) Continuation of application No. PCT/CN2024/096788, filed on May 31, 2024.

(30) Foreign Application Priority Data
Jul. 14, 2023 (CN) 202310868910.0

(51) Int. Cl. A61K 31/519; A61K 31/357; A61P 33/06

(52) U.S. Cl. CPC A61K 31/519; A61K 31/357; A61P 33/06

(57) ABSTRACT

The invention provides a transcription factor PfAP2-O5 inhibitor for Plasmodium falciparum, a pharmaceutical composition, and a drug for treating malaria resistant to artemisinin and an analog thereof. Disclosed in the present invention is new use of a triazoloquinoline derivative or a pharmaceutically acceptable salt or hydrate thereof. The triazoloquinoline derivative can inhibit the expression of an invasion gene pfap2-o5, block the invasion of Plasmodium falciparum into red blood cells, affect its growth and development, and achieve the effect of killing Plasmodium falciparum. Therefore, the triazoloquinoline derivative can be used as an inhibitor for pfap2-o5, a drug for inhibiting Plasmodium falciparum, or a drug for preventing or treating malaria.

### CROSS-REFERENCE TO RELATED APPLICATION

This application is a continuation of international application of PCT application serial no. PCT/CN2024/096788, filed on May 31, 2024, which claims the priority benefit of China application no. 202310868910.0 filed on Jul. 14, 2023. The entirety of each of the above mentioned patent applications is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Dec. 23, 2025, is named 161788-US-Sequence List and is 7,337 bytes in size.

### TECHNICAL FIELD

The present invention relates to the technical field of pharmaceuticals, and more particularly, to use of a triazoloquinoline derivative in the preparation of a drug for preventing or treating malaria.

### RELATED ART

Malaria is a parasitic disease caused by infection with the protozoan malaria parasites, capable of seriously threatening human health and life safety. Its morbidity and mortality rates remain stubbornly high among infectious diseases. According to the “World Malaria Report” released by WHO (World Health Organization) in 2021, malaria is prevalent in 87 countries and regions worldwide. In 2020, there were 241 million new cases and about 627,000 patient deaths.

The life cycle of malaria parasites is complex, mainly including the sexual reproduction stage in the definitive host body and the asexual reproduction stage in the intermediate host body. In the human body, malaria parasites (sporozoites) first invade the liver, and undergo a round of schizogonic asexual proliferation. Released merozoites enter the blood and invade red blood cells, and begin a schizogony stage inside the red blood cells. This stage is the period when malaria clinically develops, and the invasion of merozoites into red blood cells is the key link in the pathogenesis of malaria.

Currently, drug therapy serves as the main means of preventing and treating malaria, among which artemisinin-based combination therapies have effectively curbed the global spread of malaria. However, with the long-term and widespread use of artemisinin-based drugs, malaria parasite strains with reduced sensitivity to them have gradually appeared in Southeast Asia and even Africa. The emergence and global spread of such malaria parasite strains have posed severe challenges to strategies of the world's malaria prevention and control and elimination. Therefore, finding new targets for antimalarial drugs, especially developing safe and effective transmission-blocking drugs targeting the key link of the pathogenesis of malaria parasites, is of vital importance to curbing the spread of malaria.

### SUMMARY OF INVENTION

The present invention provides a new drug for preventing or treating malaria in order to overcome the above-mentioned shortcomings.

Another objective of the present invention is to provide new use of a triazoloquinoline derivative.

The above objectives of the present invention are implemented through the following technical solutions:

Use of a triazoloquinoline derivative or a pharmaceutically acceptable salt or hydrate thereof in the preparation of a transcription factor PfAP2-O5 inhibitor for Plasmodium falciparum, the triazoloquinoline derivative having a structure represented by formula (I):

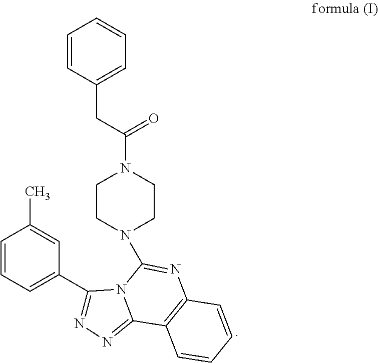 [SMILES: Cc1cccc(-c2nnc3c4ccccc4nc(N4CCN(C(=O)Cc5ccccc5)CC4)n23)c1]

The inventors conducted in-depth research on the mechanism of invasion of Plasmodium falciparum into red blood cells and discovered for the first time the function of PfAP2-O5 in positively regulating invasion into red blood cells. The triazoloquinoline derivative represented by formula (I) can inhibit the expression of the invasion gene pfap2-o5, block the invasion of Plasmodium falciparum into red blood cells, affect its growth and development, and achieve the effect of killing Plasmodium falciparum.

The PfAP2-O5 inhibitor of the present invention refers to a compound that achieves the effect of killing Plasmodium falciparum by inhibiting the function of AP2-O5.

Meanwhile, in in vitro experiments, the triazoloquinoline derivative represented by formula (I) showed good inhibitory effects on the growth of Plasmodium falciparum, while showing almost no toxicity to mammalian cells, making drug development possible. The triazoloquinoline derivative also has a good killing effect on resistant Plasmodium falciparum strains, is very helpful in delaying the generation and spread of drug resistance, and will also make antimalarial treatment more efficient.

Use of a triazoloquinoline derivative or a pharmaceutically acceptable salt or hydrate thereof in the preparation of a drug for inhibiting Plasmodium falciparum; the triazoloquinoline derivative having a structure represented by formula (I):

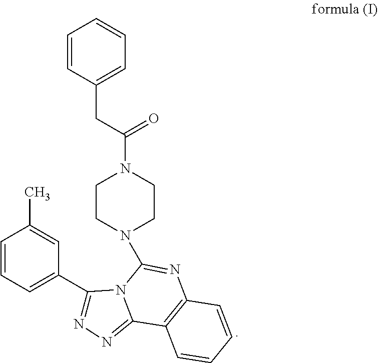 [SMILES: Cc1cccc(-c2nnc3c4ccccc4nc(N4CCN(C(=O)Cc5ccccc5)CC4)n23)c1]

Use of a triazoloquinoline derivative or a pharmaceutically acceptable salt or hydrate thereof in the preparation of a drug for preventing or treating malaria caused by Plasmodium falciparum, the triazoloquinoline derivative having a structure represented by formula (I):

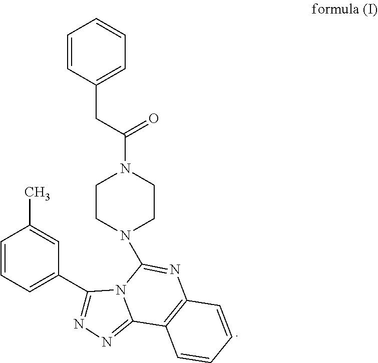 [SMILES: Cc1cccc(-c2nnc3c4ccccc4nc(N4CCN(C(=O)Cc5ccccc5)CC4)n23)c1]

In the present invention, the “pharmaceutically acceptable salt” includes a salt formed by the triazoloquinoline derivative and an inorganic acid or an organic acid.

The inorganic acid is, for example, hydrochloric acid, hydrobromic acid, sulfuric acid, phosphoric acid, nitric acid, and the like. The organic acid is, for example, acetic acid, glycolic acid, propionic acid, lactic acid, pyruvic acid, malonic acid, succinic acid, maleic acid, fumaric acid, malic acid, tartaric acid, citric acid, methanesulfonic acid, ethanesulfonic acid, benzenesulfonic acid, p-toluenesulfonic acid, cyclic acid, cyclic salicylic acid, p-aminosalicylic acid, and the like.

In the present invention, the “hydrate” refers to a salt containing water molecules bound in a certain ratio as a component of a crystal.

A pharmaceutical composition, taking a triazoloquinoline derivative including a structure represented by formula (I) or a pharmaceutically acceptable salt or hydrate thereof as an active ingredient, and a pharmaceutically acceptable carrier, diluent, or excipient as an adjuvant;

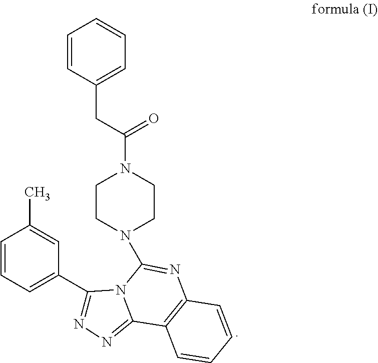 [SMILES: Cc1cccc(-c2nnc3c4ccccc4nc(N4CCN(C(=O)Cc5ccccc5)CC4)n23)c1]

During the preparation of these compositions, the active ingredient is usually mixed with an excipient, diluted with an excipient, or encapsulated in a carrier that can be in the form of a capsule or sachet. When the excipient serves as a diluent, it may be a solid, semisolid, or liquid material that acts as an excipient, carrier, or medium for the active ingredient.

Examples of suitable excipients include lactose, glucose, sucrose, sorbitol, mannitol, starch, acacia gum, calcium silicate, microcrystalline cellulose, polyvinylpyrrolidone, cellulose, water, syrup, and methylcellulose. In addition, the composition can include lubricants (such as talc, magnesium stearate, and mineral oil), wetting agents, emulsifying agents, suspending agents, preservatives (such as methyl hydroxybenzoate and propyl hydroxybenzoate), sweetening agents, or corrective agents.

Therefore, the pharmaceutical composition can be prepared into various known dosage forms; preferably, the pharmaceutical composition is prepared in an oral dosage form or an injection dosage form.

The oral dosage form includes solid dosage forms such as tablets, dripping pills, rapid-release dripping pills, capsules (such as liquid gel capsules and solid gel capsules), granules, or powders. The tablets may be compressed tablets, trituration tablets, enteric-coated tablets, sugar-coated tablets, film-coated tablets, or multi-layer compressed tablets.

Suitable adjuvant types for the preparation of the tablets include, but are not limited to, binders, lubricants, diluents, disintegrants, colorants, flavoring agents, glidants, or melting agents. The oral dosage form may also be a liquid oral dosage form, such as an aqueous solution, an emulsion, a suspension solution, a solution and/or suspension solution made from non-effervescent granules, or an effervescing agent made from effervescent granules. Suitable adjuvant types for the preparation of the liquid oral dosage forms include solvents, preservatives, emulsifying agents, suspending agents, diluents, sweetening agents, melting agents, colorants, or flavoring agents.

The injection dosage form includes: injection solution (conventional), lyophilized powder injection, powder injection (conventional), tablets for injection, and the like.

Specifically, the pharmaceutical composition may be in the form of tablets, pills, powders, elixirs, suspensions, emulsions, solutions, syrups, soft and hard gelatin capsules, suppositories, sterile injectable solutions, and sterile packaged powders.

By means of the Plasmodium falciparum growth inhibition experiment, with artemisinin as a control, an IC50 value on the Plasmodium falciparum growth inhibition of the present invention reached 2.63 μM, showing a good growth inhibition function.

The pharmaceutical composition may take a triazoloquinoline derivative of a structure represented by formula (I) or a pharmaceutically acceptable salt or hydrate thereof as a sole active ingredient, and may also take a known artemisinin-based compound as a co-active ingredient.

Preferably, the active ingredient further includes an artemisinin-based compound. The artemisinin-based compound has been demonstrated to exhibit a therapeutic effect against malaria. More preferably, the artemisinin-based compound is a dihydroartemisinin-based compound. The dihydroartemisinin-based compound has better effects on multiple drug-resistant Plasmodium falciparum than general artemisinin-based compounds.

Preferably, in the composition, a molar ratio of the triazoloquinoline derivative of the structure represented by formula (I) or the pharmaceutically acceptable salt or hydrate thereof to the artemisinin-based compound is 1:500 to 500:1.

Preferably, the pharmaceutical composition is prepared in an oral dosage form or an injection dosage form.

Use of the pharmaceutical composition in the preparation of a drug for preventing and/or treating malaria.

Preferably, use of the pharmaceutical composition in the preparation of a drug for treating malaria resistant to artemisinin and an analog thereof.

Compared with the prior art, the present invention has the following beneficial effects:

The present invention provides new use of a triazoloquinoline derivative or a pharmaceutically acceptable salt or hydrate thereof. The triazoloquinoline derivative can inhibit the expression of the invasion gene pfap2-o5, block the invasion of Plasmodium falciparum into red blood cells, affect its growth and development, and achieve the effect of killing Plasmodium falciparum. Therefore, the triazoloquinoline derivative can be used as an inhibitor for PfAP2-O5, a drug for inhibiting Plasmodium falciparum, or a drug for preventing or treating malaria. Based on the above findings, the present invention provides a new pharmaceutical composition. The pharmaceutical composition can use a triazoloquinoline derivative as an active ingredient alone, or can be combined with an artemisinin-based compound as a co-active ingredient. The pharmaceutical composition can be used for preparing a drug for treating malaria, especially a drug for treating malaria resistant to artemisinin and an analog thereof.

### BRIEF DESCRIPTION OF DRAWINGS

FIG. 1A shows the WB identification results of a PfAP2-O5 knockdown Plasmodium falciparum strain, where the expression level of a PfAP2-O5 protein is reduced after pfap2-o5 gene knockdown; FIG. 1B shows the growth curve test of the pfap2-o5 gene knockdown Plasmodium falciparum strain.

FIG. 2 shows ChIP-seq analysis of genome-wide distribution of PfAP2-O5, and the types of PfAP2-O5-binding target genes, most of which belong to invasion gene families.

FIG. 3A, FIG. 3B and FIG. 3C are schematic diagrams showing the inhibitory effect of a triazoloquinoline derivative on the expression of invasion genes. FIG. 3A shows using RNA-Seq technology to determine the expression of invasion genes of the PfAP2-O5 knockdown Plasmodium falciparum strain; FIG. 3B shows the expression of invasion genes after inhibition of the function of the PfAP2-O5 by the triazoloquinoline derivative; and FIG. 3C shows the inhibitory effects of PfAP2-O5 knockdown and treatment with the triazoloquinoline derivative on the expression of invasion genes.

FIG. 4 is a schematic diagram showing the quantitative growth inhibitory effect of a triazoloquinoline derivative on in vitro culture of Plasmodium falciparum, showing that the IC50 of the quantitative growth inhibitory effect of the triazoloquinoline derivative on in vitro culture of Plasmodium falciparum was 2.63 μM, and the growth of Plasmodium falciparum was determined using the 3-day SYBR GreenI cell growth assay.

FIG. 5 is a schematic diagram showing the cytotoxic effects of the triazoloquinoline derivative on the human renal epithelial cell line 293T and the human liver cancer cell line HepG2. It also presents the selectivity index between the antiplasmodial efficacy of the small-molecule inhibitors and their toxicity to different cell lines, with cell growth assessed using a 3-day CCK-8 assay.

FIG. 6 shows the killing effect of a triazoloquinoline derivative as a combination drug with dihydroartemisinin against resistant Plasmodium falciparum strains, where the ring-stage survival assay was used to clarify the growth inhibitory effect of the drug combination against resistant Plasmodium falciparum strains.

### DESCRIPTION OF EMBODIMENTS

The technical solutions in the embodiments of the present invention will be described clearly and completely below, but the implementation of the present invention is not limited thereto.

Unless otherwise specified, the reagents, methods, and equipment used in the present invention are conventional reagents, methods, and equipment in the technical field.
1. Triazoloquinoline derivatives of the structure represented by formula (I): purchased from ChemDiv ID. V003-8956
2. Plasmodium falciparum (3D7_G7): purchased from ATCC (American Type Culture Collection)
3. 293T cell line: purchased from ATCC
4. HepG2 cell line: purchased from ATCC
5. SYBR Green I: purchased from Invitrogen Cat. No. S-7585
6. Trypsin-EDTA: purchased from Gbico Cat. No. 25200-072
7. DMEM: purchased from Gbico Cat. No. 11965-092
8. FBS: purchased from Gbico Cat. No. 10082-147
9. Double-stranded DNA quantitation assay kit: purchased from ThermoFisher Cat. No. P7581
10. Protein A/G magnetic beads: purchased from ThermoFisher Cat. No. 88803
11. Mouse monoclonal antibody: purchased from Sigma Cat. No. SAB4800032
12. 37% formaldehyde solution: purchased from Sigma Cat. No. 47608-250ML-F
13. 96-well plate: purchased from Corning
14. Red blood cells: purchased from Shanghai Blood Center
15. Dihydroartemisinin (CAS: 71939-50-9)
16. The components of various buffers are as follows:

### (1) Lysis Buffer

| | |
|---|---|
| Reagent (stock solution) | Final concentration |

| | | |
|---|---|---|
| 1M Hepes pH 7.9 | 10 | mM |
| 0.5M EDTA pH 8.0 | 0.1 | mM |
| 0.5M EGTA pH 8.0 | 0.1 | mM |
| 2M KCl | 10 | mM |
| 1M DTT (add before use) | 1 | mM |

| | |
|---|---|
| 100× Protease inhibitor (add before use) | 1x |

### (2) Sonication Buffer

| Reagent (stock solution) | Final concentration |
|---|---|
| 10% SDS | 0.1% |
| 0.5M EDTA pH 8.0 | 1 mM |
| 1M Tris-HCl pH 8.0 | 10 mM |

### (3) ChIP Dilution Buffer

| Reagent (stock solution) | Final concentration | |
|---|---|---|
| 10% SDS | 0.01% | |
| 100% Triton X-100 | 1.1% | |
| 5M NaCl | 150 | mM |
| 0.5M EDTA pH 8.0 | 1.2 | mM |
| 1M Tris-HCl pH 8.0 | 16.7 | mM |

### (4) Low-Salt Immune Complex Wash Buffer

| Reagent (stock solution) | Final concentration | |
|---|---|---|
| 10% SDS | 0.1% | |
| 100% Triton X-100 | 1% | |
| 5M NaCl | 150 | mM |
| 0.5M EDTA pH 8.0 | 2 | mM |
| 1M Tris-HCl pH 8.0 | 20 | mM |

### (5) High-Salt Immune Complex Wash Buffer

| Reagent (stock solution) | Final concentration | |
|---|---|---|
| 10% SDS | 0.01% | |
| 100% Triton X-100 | 1% | |
| 5M NaCl | 500 | mM |
| 0.5M EDTA pH 8.0 | 2 | mM |
| 1M Tris-HCl pH 8.0 | 20 | mM |

### (6) Lithium Chloride Immune Complex Wash Buffer

| Reagent (stock solution) | Final concentration | |
|---|---|---|
| 10% NP-40 | 1% | |
| 10% deoxycholic acid | 1% | |
| 5M LiCl | 250 | mM |
| 0.5M EDTA pH 8.0 | 1 | mM |
| 1M Tris-HCl pH 8.0 | 10 | mM |

### (7) TE Buffer

| Reagent (stock solution) | Final concentration |
|---|---|
| 1M Tris-HCl pH 8.0 | 10 mM |
| 0.5M EDTA pH 8.0 | 1 mM |

### (8) Elution Buffer

| Reagent (stock solution) | Final concentration |
|---|---|
| 10% SDS | 1% |
| 1M NaHCO₃ | 100 mM |

### Example 1 Culture of Plasmodium falciparum

Plasmodium falciparum was cultured in RPMI 1640 complete culture medium at 37° C. in an incubator (5% CO₂, 5% O₂, and 90% N₂).

0.5 L RPMI 1640 complete culture medium contained: 5.22 μg RPMI-1640 powder; 1.0 μg sodium bicarbonate; 5 μg AlbmaxI; 2.98 μg 25 mM HEPES; 13.6 mg hypoxanthine; 10 mg gentamicin sulfate.

### Example 2 Construction of Transgenic Plasmodium falciparum Strain: Pfap2-o5 Conditional Knockdown Plasmodium falciparum Strain

In this experiment, a CRISPR/Cas9 gene editing system was used to clone the guide RNA into a vector to construct a pfap2-o5 conditional knockdown Plasmodium falciparum strain.

The detailed construction procedures were as follows:
1. Construction of transgenic Plasmodium falciparum strain knockdown vector (designated as pL6cs-ap2-o5-glmS)

Using the CRISPR/Cas9 gene editing system to clone the guide RNA into the vector was as follows in detail:

(1) Design of gRNA

The sequence of the pfap2-o5 gene of Plasmodium falciparum 3D7_G7 strain was downloaded from a PlasmoDB website (Plasmodium falciparum PF3D7_1449500,715aa, PfAP2-O5); this sequence is also available in the NCBI database: Sequence ID: XM_001348609.1). By means of BLAST (Basic Local Alignment Search Tool) alignment, a specific sequence of 20 bp in length was selected as a guide RNA (gRNA). The sequence was as follows: 5′-ACCAAGTCATATGTCATTAA-3′ (SEQ ID NO.1).

### (2) Design of Primers

Primers were designed using SnapGene software:

P1:
(SEQ ID NO. 2)
5′-GCCCTAGTCTAGGGcGCGCCCAATTTTAATAACAATTTGGCG-3′;
P2:
(SEQ ID NO. 3)
5′-CAAGTAAAATAATTGGTCTTTAAGTGACATATGACTTGGTAGTT
TACTG-3′;
P3:
(SEQ ID NO. 4)
5′-AGCGGCCGCAAAAAAAATAAAAAAAAAAAAAAAAAAAAAAA
AAAAAAAATATATATATTATAC-3′;
P4:
(SEQ ID NO. 5)
5′-TTTTTTACAAAATGCTTAAGAACTAGCGTATGTGATAACTTAG-3′,

used to amplify the homologous arms of the pfap2-o5 gene and introduce synonymous mutation sites, the introduction of synonymous mutation sites being intended to prevent self-cleavage of the plasmid by the gRNA.

P7:
(SEQ ID NO. 6)
5′-TATTTTACTTGGACAAATTAGGTACCGGAAGTGGTTCTGG-3′;
P8:
(SEQ ID NO. 7)
5′-TTTATTTTTTTTGCGGCCGCGCGGCCGCGTCCCCTC-3′,

used to amplify the exogenous green fluorescent protein (GFP) gene.

### (3) Gene Amplification

A genomic DNA of the Plasmodium falciparum: 3D7_G7 strain was extracted. The left and right homologous sequences of the pfap2-o5 gene were amplified using primers P1+P2 and P3+P4, respectively, and mutation sites were introduced. A glmS ribozyme sequence was amplified using primers P7+P8 (the glmS sequence was obtained from the literature report: doi:10.1371/journal.pone.0073783).

### (4) Construction of Ligation Product

The left and right homologous sequences were respectively ligated to the glmS ribozyme sequence using primers, yielding a ligation product.

The 3′ end of the pfap2-o5 gene was tagged with the glmS ribozyme sequence.

The reaction system was as follows: 2×Phanta Max Master Mix (Dye Plus): 25 μL; primer P1 (10 μM): 2 μL; primer P4 (10 μM): 2 μL; the left and right homologous sequences and the glmS ribozyme sequence: 1 μL each; ddH₂O: to a final volume of 50 μL.

PCR conditions were as follows: 95° C. for 3 minutes, 95° C. for 15 seconds, 65° C. for 15 seconds, 65° C. for 30 s/kb, 65° C. for 5 minutes, with a total of 30 cycles; and 72° C. for 10 minutes.

### (5) Construction of Knockdown Vector

Plasmid pL6cs (constructed in our laboratory; for detailed sequence information, see the literature report: doi: 10.3389/fmicb.2020.625862), carrying the resistance gene for a screening drug dihydrofolate reductase inhibitor (WR), was double-digested with XhoI and AvrII. The product was recovered and purified using a gel extraction kit. The gRNA fragment was ligated into the purified vector using DNA ligase. The ligation product was transformed into Escherichia coli XL-10 competent cells. The cells were inoculated onto LB agar plates containing 100 μg/mL ampicillin and cultured overnight. Single colonies were picked and cultured overnight at 34° C. with shaking at 200 rpm/s. The plasmids were extracted and sequenced.

The plasmids linked to gRNA were confirmed by sequencing and double-digested with AscI and AflII. The product was recovered and ligated to the homologous arms containing the glmS ribozyme sequence for transformation. Single colonies were picked for culture. The correct plasmids were identified by PCR for sequencing. The strain was stored at −80° C.

The resulting transgenic Plasmodium falciparum strain knockdown vector was designated as pL6cs-ap2-o5-glmS.

2. Construction of Pfap2-o5 Conditional Knockdown Plasmodium falciparum Strain

A transgenic Plasmodium falciparum strain, pfap2-o5 conditional knockdown Plasmodium falciparum strain, was constructed using electroporation combined with drug selection.

### (1) Preparation of Plasmids for Electroporation

The strains confirmed as correct by sequencing were inoculated onto LB agar plates containing 100 μg/mL ampicillin and cultured overnight. The next day, single clones were picked into an LB culture solution for shaking culture at 34° C. and 200 rpm/s for 8 hours. The mixture was diluted 1:1000 into 400 mL of an LB culture solution. After shaking culture for 14 hours, bacterial cells were collected, and the plasmids were collected using a plasmid DNA purification kit.

100 μg of the transgenic Plasmodium falciparum strain knockdown vector pL6cs-ap2-o5-glmS plasmid constructed above and 100 μg of the plasmid pUF1-Cas9 carrying the resistance gene for a screening drug blasticidin (BSD) and expressing Cas9 protein (the plasmid was constructed in our laboratory; for detailed sequence information, see the literature report: doi: 10.3389/fmicb.2020.625862) were taken. A 3M CH₃COONa (pH 5.2) solution with a volume one-tenth that of the plasmid solution was added. After even mixing, anhydrous ethanol with a volume 2.5 times that of the plasmid solution was added. The mixed solution was thoroughly mixed well, and centrifuged at 13,000×g for 30 minutes. The supernatant was discarded. The pellet was washed twice with 75% ethanol, air-dried in a super clean bench, and dissolved in 150 μL of sterile water.

(2) Electroporation and Screening of Transgenic Plasmodium falciparum Strains

The electroporator was set to the following conditions: electroporation parameters: 310 V, 950 μF, resistance ∞, and a 2 mm gap. An equal volume (150 L) of 2× cell mixed solution was added to the prepared plasmid and mixed well. 150 μL of red blood cells were then added. After even mixing, the mixture was transferred to an electroporation cuvette, which was placed in an electroporator for electroporation. After electroporation, the mixed solution was transferred to a culture flask. An appropriate amount of culture solution was added, and purified schizont-stage Plasmodium falciparum was then added.

Culture was performed in a 37° C. tri-gas incubator. Starting from the second day, the culture medium was replaced daily with fresh culture medium. On the fourth day, when the electroporation density was approximately 10% (parasitemia, the proportion of red blood cells infected with Plasmodium falciparum among all red blood cells counted in a certain field of view (approximately 5000 cells)), 200 μL of fresh red blood cells were supplemented, the culture solution was replaced, and screening drugs WR and BSD were added. The screening drugs could kill the wild-type Plasmodium falciparum, while the successfully genetically modified Plasmodium falciparum was not affected by the drugs because they carried the plasmids expressing drug-resistant genes. Therefore, transgenic Plasmodium falciparum could be screened out.

After the screening drugs were added until most of the wild Plasmodium falciparum were killed, the replacement of the culture solution and the addition of the drugs were performed every 3 to 4 days to maintain the culture. Smear test under microscope was started on the 21st day after transfection, and transgenic Plasmodium falciparum strains were obtained within 3 to 6 weeks.

Based on above, a transgenic Plasmodium falciparum strain, pfap2-o5 conditional knockdown Plasmodium falciparum strain, was constructed and obtained.

### 3. Gradient Dilution Cloning

The transgenic Plasmodium falciparum strain (pfap2-o5 conditional knockdown Plasmodium falciparum strain) obtained by genetic modification was an integrated strain and required gradient dilution cloning. That is, a completely integrated monoclonal strain was obtained by screening through limiting dilution. This experiment was performed in a 96-well cell culture plate. Through gradient dilution, the parasite-infected red blood cells were diluted to theoretical concentrations of 10, 1, 0.5, and 0.1 parasites per 1 mL, respectively. The detailed experimental procedures were as follows:
(1) the transgenic Plasmodium falciparum strains were synchronized with sorbitol to obtain relatively homogeneous ring-stage parasites;
(2) smear test under microscope was performed during the trophozoite stage, and the initial parasitemia Px was strictly counted;
(3) trophozoite-stage cultures were collected and centrifuged at 2000 rpm at room temperature for 5 minutes, and the supernatant was discarded to obtain approximately 200 μL of packed infected red blood cells (iRBCs);
(4) 800 μL of fresh culture medium was added to the iRBCs, and gently mixed well, and the mixture was transferred to a 1.5 mL centrifuge tube, bringing the final volume to 1 mL;
(5) the mixture was centrifuged at 1500 rpm for 2-3 minutes at room temperature; the supernatant was discarded; 50 μL of the iRBCs were taken to a new centrifuge tube for later use;
(6) according to the formula 50 μL×Px=1%×(50 μL+Vx), 50 μL of the infected red blood cells were diluted to the parasitemia of 1%, yielding culture A (Vx is the volume of fresh red blood cells added), and the parasitemia after dilution was confirmed by smear test under microscope;
(7) the fresh culture medium was preheated; according to the principle of gradient dilution, the infected red blood cells with the parasitemia of 1% prepared above were sequentially diluted in proportion so that each centrifuge tube corresponds to one gradient:

Nine 15 mL centrifuge tubes were prepared and sequentially numbered 1, 2, 3, . . . , 9; 10 mL of fresh culture medium was added to tube 1; 9 mL of fresh culture medium was added to tubes 2-7 and 9; 5 mL of fresh culture medium was added to tube 8; all centrifuge tubes were preheated in a 37° C. incubator for later use. 100 μL of culture A with the parasitemia of 1% in (6) was taken and added to the above tube 1 to obtain culture B with 1×10⁶ iRBCs/mL;
1) 1 mL of culture B was taken to tube 2 to obtain culture C with 1×10⁵ iRBCs/mL;
2) 1 mL of culture C was taken to tube 3 to obtain culture D with 1×10⁴ iRBCs/mL;
3) 1 mL of culture D was taken to tube 4 to obtain culture E with 1×10³ iRBCs/mL;
4) 1 mL of culture E was taken to tube 5 to obtain culture F with 1×10² iRBCs/mL;
5) 1 mL of culture F was taken to tube 6 to obtain culture G with 10 iRBCs/mL;
6) 1 mL of culture G was taken to tube 7 to obtain culture H with 1 iRBC/mL;
7) 5 mL of culture H was taken to tube 8 to obtain culture I with 0.5 iRBC/mL;
8) 1 mL of culture H was taken to tube 9 to obtain culture J with 0.1 iRBC/mL.
(8) the cultures G, H, I, and J with different gradients obtained above were transferred to 96-well cell culture plates, respectively, with 200 μL/well, and finally, each row of 12 wells of the 96-well plates corresponded to one gradient;
(9) in the later stage of culture, fresh culture medium was replaced every 2 days, and fresh red blood cells (2 L/well) were added every 6 days to maintain the culture;

After about 2 weeks, smear test under microscope was performed. Once live parasites were detected, the culture could be gradually scaled up using 24-well, 12-well, and 6-well plates; after the genome was tested as correct, a completely integrated monoclonal pfap2-o5 transgenic Plasmodium falciparum strain (pfap2-o5 conditional knockdown Plasmodium falciparum strain) was obtained, preserved, and used for subsequent experiments.

### Example 3 Identification and Growth and Development Testing of Pfap2-o5 Transgenic Plasmodium falciparum Strain (Pfap2-o5 Conditional Knockdown Plasmodium falciparum Strain)

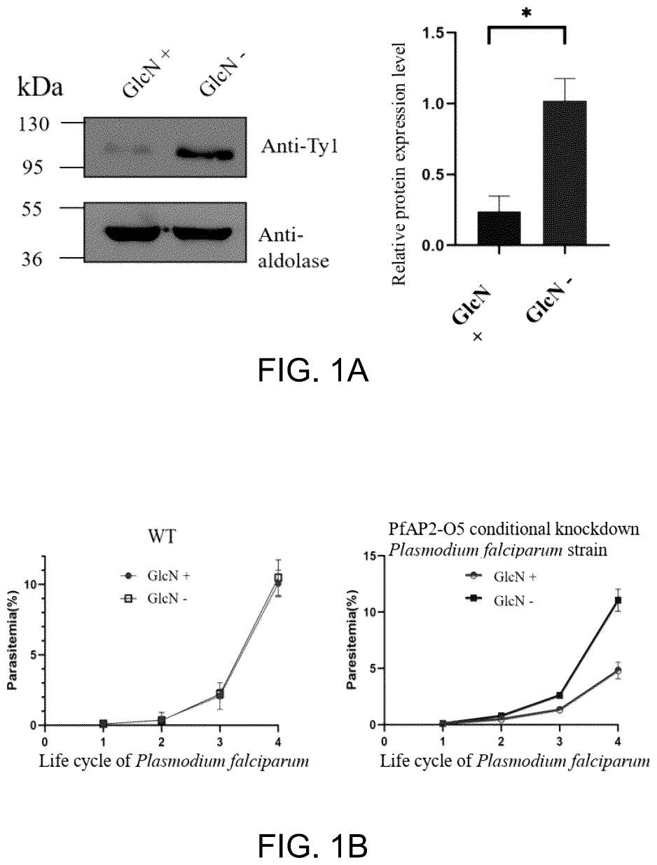

The ring (10-15 hours), trophozoite (28-32 hours), and schizont (40-44 hours) stage cultures of the pfap2-o5 transgenic Plasmodium falciparum strain (pfap2-o5 conditional knockdown Plasmodium falciparum strain) were collected. Red blood cells infected with Plasmodium falciparum were lysed with 0.15% saponin to collect protein samples; after separation by SDS-PAGE (Sodium dodecyl sulfate polyacrylamide gel electrophoresis), the proteins were transferred to nitrocellulose membranes by semi-dry transfer under parameters of 0.1 A for 2 hours. After the transfer was completed, an appropriate volume of blocking solution (5% skim milk powder, the skim milk powder being dissolved in PBST solution) was added for blocking at room temperature for 2 hours. Incubation with a primary antibody (mouse-derived anti-Ty1 antibody): the primary antibody (anti-Ty1) was diluted 1:1000 with 5% skim milk powder, and the membrane was sealed and incubated overnight at 4° C. Incubation with a secondary antibody: the secondary antibody (goat anti-mouse) was diluted 1:5000 with 5% skim milk powder, and the membrane was incubated at room temperature for 2 hours. Color development: equal volumes of color development solutions A and B were taken and mixed well (protected from light), a certain amount of color development solution was taken and dropwise added to the membrane, and the exposed images were captured and saved. The results are shown in FIG. 1A. FIG. 1A shows western blot identification of the PfAP2-O5 conditional knockdown Plasmodium falciparum strain before and after the addition of 5 mM glucosamine (GlcN), indicating that PfAP2-O5 was effectively knocked down.

For the growth curve tests of the pfap2-o5 conditional knockdown Plasmodium falciparum strain, the Plasmodium falciparum strains were synchronized several times to the ring stage; the initial parasitemia was determined by strictly counting; the culture was divided into two flasks (one added with the corresponding concentration of GlcN, and the other serving as an untreated control), and the initial parasitemia of each flask was approximately 0.1%. Both cultures were cultured continuously for four life cycles. At the mid to late stages of each life cycle, smear test under microscope was performed, and the parasitemia was counted (the culture medium was replaced if the parasitemia was high, with the drug replenished in the treated group). The parasitemia of the Plasmodium falciparum strains in the treated and untreated groups was recorded, and growth curves were drawn. The results are shown in FIG. 1B. FIG. 1B shows the growth curve tests of the wild-type Plasmodium falciparum strain and the pfap2-o5 conditional knockdown Plasmodium falciparum strain, indicating that the growth of Plasmodium falciparum was significantly inhibited after conditional knockdown of pfap2-o5.

### Example 4 Chromatin Immunoprecipitation (ChIP) Sequencing

Chromatin immunoprecipitation (ChIP) sequencing utilizes the specificity of the antigen-antibody reaction to truly reflect the genome-wide distribution of AP2-O5-bound target genes at the chromatin level, while also reflecting the binding of the protein to the target gene. The detailed procedures were as follows:

The synchronized schizont stage cultures were collected, 1% formaldehyde solution was added, and the cultures were incubated at 37° C. for 10 minutes. Pre-cooled 0.125 M glycine solution was added, and the cultures were incubated on ice for 5 minutes. The cells were centrifuged at 2500 rpm and 4° C. for 5 minutes, the supernatant was discarded, 30 mL of 1×PBS was added to resuspend the red blood cells, 0.15% saponin was added to lyse the red blood cells, and the parasites were collected. 2 mL of pre-cooled Lysis Buffer was added to resuspend the parasites, and the mixture was transferred to a pre-cooled grinding tube for ice bath for 30 minutes. 200 μL of ultrasonic buffer was added to resuspend the pellet, and liquid phase ultrasonication was performed with the following set parameters: duty cycle 5%, peak incident power 75 W, 200 cycles, temperature 7° C., and time 30 minutes. 10 L of the sonicated mixed solution was taken; 90 μL of ChIP dilution buffer, 4 μL of 5 M NaCl, and 2.5 μL of proteinase K were added; the mixture was mixed well and incubated at 50° C. for 2 hours, 2 μL of RNase A was added, and the mixture was incubated at 37° C. for 45 minutes. Extraction was performed with the QIAGEN PCR kit, and the sonication effect was tested with 2% agarose gel electrophoresis. Sonication products with an ideal sonication effect were centrifuged at 14,000 rpm for 10 minutes at 4° C.; the supernatant was then collected; ChIP dilution buffer with a volume 10 times that of the supernatant was added. Protein A/G magnetic beads were added to the above solution; the mixed solution was incubated at 4° C. with shaking for 2 hours; the supernatant was transferred to a 1.5 mL centrifuge tube; the corresponding antibody and protein A/G magnetic beads were added; the mixture was incubated overnight at 4° C. The antibody/magnetic bead/chromatin complex was washed with different buffers added in the following order:
A: Low-salt immune complex wash buffer: 1 mL per tube, rotary washing at 4° C. for 5 minutes;
B: High-salt immune complex wash buffer: 1 mL per tube, rotary washing at 4° C. for 5 minutes;
C: Lithium chloride immune complex wash buffer: 1 mL per tube, rotary washing at 4° C. for 5 minutes;
D: TE buffer: 1 mL per tube, rotary washing at 37° C. for 5 minutes, twice;
200 μL of elution buffer was added for elution. Rotary incubation was performed at 37° C. for 30 minutes. The supernatant was collected, and 180 μL of elution buffer was added to DNA tubes before immunoprecipitation. 16 μL of 5 M NaCl was added to each of the above tubes, and the mixture was mixed well and incubated overnight at 45° C. After the incubation was completed, 0.8 μL of RNase A (20 mg/mL) was added to each tube, and the mixture was incubated at 37° C. for 30 minutes. 3 μL of proteinase K was added to each tube, and the mixture was incubated at 45° C. for 2 hours. The sample DNA was extracted using the QIAGEN PCR purification kit and eluted twice with 10 μL of deionized water, letting it stand at room temperature for 10 minutes before each elution. The concentration was measured, and the library was constructed. To prepare the sequencing library, 1.5 ng of ChIP-DNA was subjected to end repair, 3′ adenylation, and adapter ligation. After purification using Agencourt AMPure XP magnetic beads, the library was amplified using the KAPA HiFi PCR kit under the following conditions: 95° C. for 1 minute, 98° C. for 10 seconds, 65° C. for 1 minute, 12 cycles, 65° C. for 5 minutes, and 4° C. for ∞. The library was sequenced on the Illumina HiSeq X Ten platform; two biological replicates were performed for each ChIP-seq dataset.

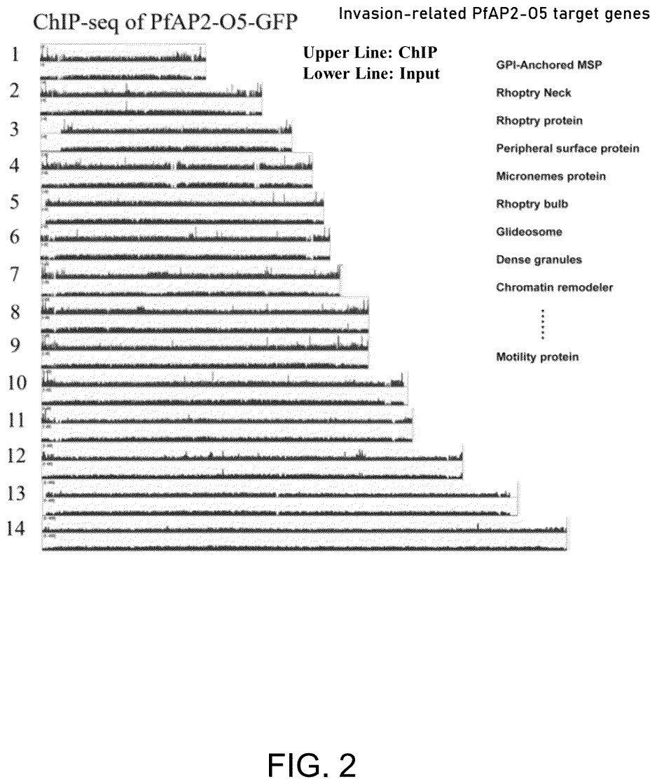

The results are shown in FIG. 2, which shows the genome-wide distribution of PfAP2-O5 protein target genes, such as invasion gene families including msps, raps, rhophs, ralp1, and ama1, proving that most PfAP2-O5 target genes are associated with invasion.

### Example 5 Transcriptome Sequencing (RNA-seq) Analysis of Effects of Triazoloquinoline Derivative on PfAP2-O5

Wild-type and pfap2-o5 conditional knockdown Plasmodium falciparum strains were treated with the triazoloquinoline derivative represented by formula (I), respectively. After strict synchronization of the Plasmodium falciparum strains, 1 M triazoloquinoline derivative was added one life cycle in advance. After one life cycle, the ring (10-15 hours), trophozoite (28-32 hours), and schizont (40-44 hours) stage cultures of the wild-type and pfap2-o5 conditional knockdown Plasmodium falciparum strains treated with the triazoloquinoline derivative were collected. Total RNA was purified using the Direct-zol RNA kit, strand-specific RNA sequencing libraries were then prepared using KAPA mRNA beads, and the RNA was fragmented. The libraries were sequenced on the Illumina HiSeq X Ten system, and the differential gene expression between the two Plasmodium falciparum strains after inhibitor treatment was analyzed.

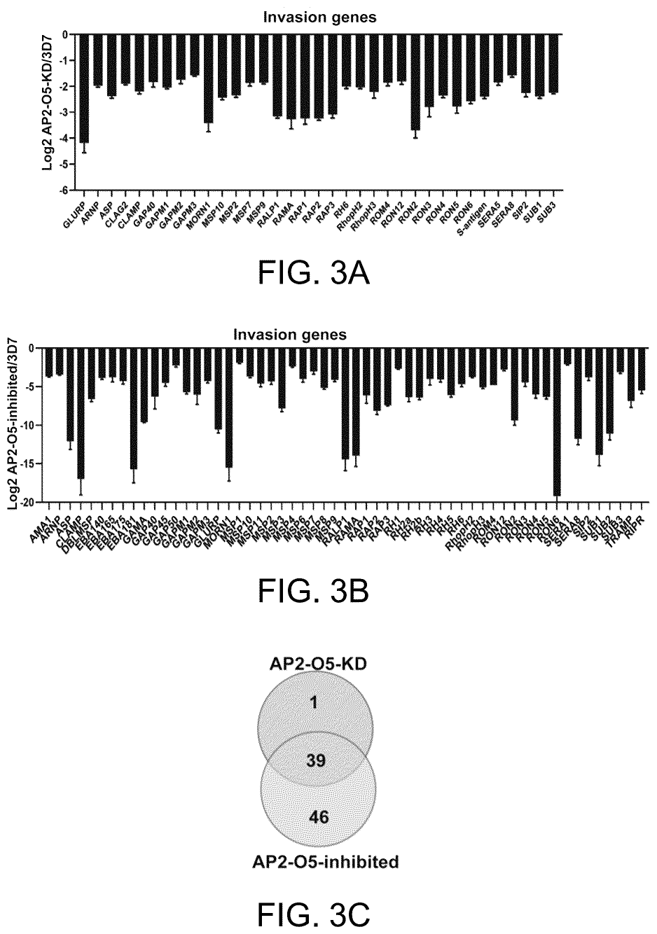

The results are shown in FIG. 3A, FIG. 3B and FIG. 3C; in FIG. 3A, RNA-Seq technology was used to determine the expression of invasion genes after conditional knockdown of PfAP2-O5; in FIG. 3B, RNA-Seq technology was used to determine the inhibitory effects on the expression of invasion genes after inhibition of the function of PfAP2-O5 by the triazoloquinoline derivative. The results show that after conditional knockdown of PfAP2-O5, invasion-related target genes were downregulated, and after the wild-type Plasmodium falciparum strains were treated with the triazoloquinoline derivative, PfAP2-O5 target genes were downregulated. For example, the expression levels of Plasmodium falciparum merozoite surface structural protein family genes, such as Rhoptry Neck, Peripheral surface protein, Rhoptry protein, Rhoptry bulb, Micronemes protein, and GPI-Anchored MSP, showed a significant decrease of more than 1.5-fold (mRNA level). (Plasmodium falciparum merozoite surface structural protein family genes mainly regulate the invasion of Plasmodium falciparum merozoites into red blood cells. The main references are as follows: DOI: 10.1016/j.chom.2017.07.003; DOI: 10.1016/j.chom.2017.05.006). FIG. 3C shows the intersection of invasion-related target genes after conditional knockdown of PfAP2-O5 and inhibition of the function of PfAP2-O5 by the triazoloquinoline derivative.

### Example 6 Determination of IC50 of In Vitro Growth Inhibition of Triazoloquinoline Derivative Against Plasmodium falciparum

100 μL of complete culture medium was added to a 96-well plate. 200 μL of a compound stock solution with a concentration of 200 μM was added to the first well. Gradient dilution (11 concentration gradients) was performed at a 1/2 ratio according to the doubling gradient dilution. Dihydroartemisinin (DHA) was used as a positive control, complete culture medium was used as a negative control, and red blood cells without Plasmodium falciparum or compounds were used as a background control.

100 μL of wild-type Plasmodium falciparum culture (1% parasitemia, 4% hematocrit) was accurately added to each well, so that finally, the parasitemia in each well was 0.5%, and the hematocrit was 2%. The final concentration gradients of the compound were 100 μM, 50 μM, 25 μM, 12.5 μM, 6.25 μM, 3.125 μM, 1.5625 μM, 0.78125 μM, 0.390625 μM, 0.195313 μM, and 0.097656 μM. All samples were set up in triplicate. After the addition was completed, the 96-well plate was placed in a 37° C. incubator (5% CO₂, 5% O₂) and cultured for a precise duration of 72 hours. After the culture was completed, 100 μL of Lysis buffer (1×SYBR Green I, 0.12 mg/mL saponin, 0.12% v/v Triton X-100, 30 mM Tris-HCl, and 7.5 mM EDTA) was added to each well. The plate was thoroughly mixed well, and incubated at room temperature in the dark for 2 hours. After the incubation was completed, a fluorescence intensity was recorded on a microplate reader with an excitation wavelength of 485 nm and an emission wavelength of 535 nm. The inhibition rate of the small molecule inhibitor on the growth of Plasmodium falciparum was calculated based on the fluorescence intensity.

$$Survival Rate = ( {Fl}_{t r e a t e d} - {Fl}_{background} ) / ( {Fl}_{control} - {Fl}_{background} )$$

where Fl_{treated}, Fl_{background}, and Fl_{control} represent the fluorescence intensity values of the candidate small molecule drug group, the red blood cell control group, and the untreated control group, respectively.

An inhibition rate of the drug was calculated based on the survival rate at different concentrations, and a growth inhibition curve was summarized as inhibition rate-drug concentration. The IC50 was calculated using SPSS 18.0 software.

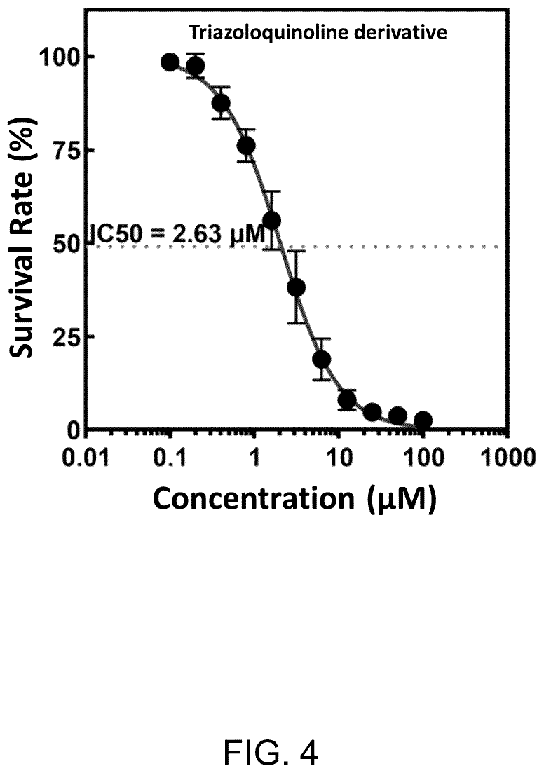

The results are shown in FIG. 4, where the IC50 of the quantitative growth inhibitory effect of the triazoloquinoline derivative on in vitro culture of Plasmodium falciparum was 2.63 μM.

### Example 7 Determination of IC50 of Cytotoxicity of Small Molecule Inhibitor

Mammalian 293T and HepG2 cells were cultured and passaged, trypsinized, and resuspended in DMEM. The cells were counted using a hemocytometer and diluted to 10⁵ cells/mL. The cell suspension was evenly added to a 96-well plate, with 100 μL per well. After the cells were subjected to attachment culture in a 37° C. incubator (5% CO₂) for 24 hours, the inhibitors of the corresponding concentrations were added. Gradient dilution (11 concentration gradients) was performed at a 1/2 ratio according to the doubling gradient dilution. Complete culture medium was used as a negative control, and DMEM was used as a background control. After the culture in a 37° C. incubator (5% CO₂) for 72 hours, 10 L of CCK8 reagent was added to each well. The plate was incubated in a 37° C. incubator (5% CO₂) for 1 hour. The absorbance at a 450 nm wavelength was read by a microplate reader, and a survival rate was calculated based on the absorbance.

$$Survival Rate = ( A {b}_{t r e a t e d} - {Ab}_{background} ) / ( {Ab}_{control} - {Ab}_{background} )$$

where Ab_{treated}, Ab_{background}, and Ab_{control} represent the absorbance values of the small molecule drug group, the DMEM control group, and the untreated control group, respectively. The inhibition rate of the drug was calculated based on the survival rate at different concentrations, and the growth inhibition curve was summarized as inhibition rate-drug concentration. The IC50 was calculated using SPSS 18.0 software.

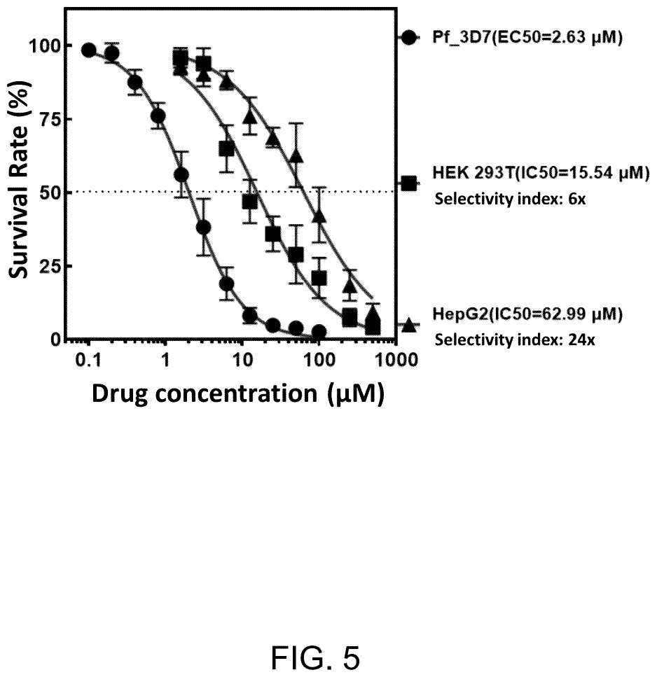

The results are shown in FIG. 5. FIG. 5 reflects that the triazoloquinoline derivative of the structure of formula (I) of the present invention has low cytotoxicity to mammalian cells and has good biosafety.

### Example 8 Pharmacokinetic Analysis of Small Molecule Inhibitor

This experiment aimed to investigate the species differences in the metabolic rates of the triazoloquinoline derivative of the structure of formula (I) in mouse, rat, and human liver microsomes.

In the microsomal metabolism assay, the inhibitor was incubated with liver microsomes from different species; the reaction was terminated at different time points; the remaining amount of the inhibitor in the samples at each time point was determined using LC-MS/MS. The percentage of parent drug remaining was calculated, and the hepatic intrinsic clearance CLᵢₙᵢₜ₍ₗᵢᵥₑᵣ₎ was finally obtained. Testosterone was used as a control in this study to verify the reliability of the experimental system. The detailed procedures were as follows:

The incubation system consisted of 0.1 M Tris-HCl buffer (pH 7.4), 1 mM MgCl₂, 1 mM NADPH (Nicotinamide Adenine Dinucleotide Phosphate), and 0.5 mg/mL microsomes, and the final reaction volume was 200 μL. 1 M inhibitor and testosterone were pre-incubated with liver microsomes from different species in a 37° C. water bath for 5 minutes. 100 μL of NADPH was added to initiate the reaction. 400 μL of stop solution was added to terminate the reaction at time points of 0 minutes, 5 minutes, 15 minutes, 30 minutes, 45 minutes, and 60 minutes. 40 μL of internal standard was added, and the mixture was vortexed for 5 minutes and centrifuged at 20,000 μg and 4° C. for 5 minutes. 100 μL of the supernatant was taken and diluted with 100 μL of water for injection and analysis. The retention time of the compound and the internal standard, chromatogram acquisition, and chromatogram integration were processed by Analyst 1.6.3 software. The compound was subjected to linear regression with a weighting coefficient of 1/X² to calculate the concentration at each time point. The natural logarithm of the percentage remaining at each time point was linearly regressed against the incubation time to calculate the in vitro elimination rate constant k. The half-life (T_{1/2}) and hepatic intrinsic clearance CLᵢₙₜ₍ₗᵢᵥₑᵣ₎ of the test article were calculated according to the following formula.

$$Half - life {T}_{1 / 2} ( min ) = \frac{0.693}{- k} ;$$

hepatic microsomal clearance C_{Lint(mic)}=0.693/half-life/mg microsomal protein per mL; hepatic intrinsic clearance CLᵢₙₜ₍ₗᵢᵥₑᵣ₎=hepatic microsomal clearance×mg microsomal protein/gram of liver×liver weight-to-body weight ratio. The liver weight-to-body weight ratios of mice, rats, and humans were 88, 40, and 20 μg/kg, respectively; the average value of mg microsomal protein/gram of liver in different species was 45. The results of mouse/rat/human microsomal metabolism of the triazoloquinoline derivative are shown in Table 1.

TABLE 1

| Species | k (min⁻¹) | T_{1/2} (min) | CLᵢₙₜ (mL/min/kg) |
|---|---|---|---|
| Mouse | 0.0432 | 16.04 | 342.144 |
| Rat | 0.0165 | 42.05 | 59.4 |
| Human | 0.0053 | 130.75 | 8.54 |

Liver microsomal metabolism results show that the inhibitor is a moderate-clearance compound in rat and human liver microsomes and a high-clearance compound in mouse liver microsomes.

### Example 9 Ring-Stage Survival Assay of Triazoloquinoline Derivative Against Resistant Plasmodium falciparum Strains

This experiment aimed to evaluate the killing effect of the triazoloquinoline derivative as a combination drug against drug-resistant Plasmodium falciparum strains using the gold-standard drug resistance assay. The detailed implementation procedures were as follows:
1. Culture of Plasmodium falciparum when the parasitemia was greater than 4% and most parasites were at the ring (R) stage, the culture was synchronized with Sorbitol for 10 minutes and vortexed for 5 seconds;
2. Synchronization with sorbitol was repeated 30-48 hours later (depending on the parasite stage);
3. Smear test under microscope after approximately 30 hours of culture: if the proportion of the mature schizont (S) stage (10-12 merozoites) was greater than 0.5%, the operation proceeded to procedure 4; if the proportion of the mature S stage (10-12 merozoites) was less than 0.5%, procedure 2 was repeated;
4. The culture was collected for enrichment of late-stage parasites (Plasmion or Percoll); smear test under microscope was performed on the enriched parasites, the proportion of the S stage must be greater than 10%, and the proportion of the R stage must be less than 10%;
5. 10 mL of culture medium and 200 μL (appropriate amount) of fresh red blood cells were added to maintain the culture for 3 hours (must be accurate);
6. After the culture was mixed well, approximately 200 μL of the culture was pipetted and centrifuged for smear test under microscope (this could be done approximately 10 minutes in advance); the parasitemia was quickly calculated (must be greater than 0.5%);
7. The culture was collected, subjected to synchronization with Sorbitol for 10 minutes, vortexed for 5 seconds, and centrifuged; the supernatant was discarded;
8. In a 24-well plate: Control group: 100 μL of DMSO (Dimethylsulfoxide) control solution was added to each well;
Experimental group: 100 μL of 7 mM DHA solution and 20 M triazoloquinoline derivative solution (dissolved in culture medium) were added to each well;
9. 900 μL of resuspended iRBC culture was added to each well; the mixture was mixed well to maintain the culture for a precise duration of 6 hours;
10. After 6 hours, the culture in each well was transferred to a 1.5 mL EP (Eppendorf) tube and centrifuged to remove the supernatant;
11. 1 mL of preheated culture medium was added for washing; the mixture was centrifuged to remove the supernatant; the washing was repeated once, for a total of two washes;
12. 1 mL of preheated culture medium was added; the mixture was mixed well and transferred to a new well plate to continue to maintain the culture for 66 hours;
13. After 66 hours of the culture, smear test under microscope was performed to calculate the parasitemia, and the survival rate was calculated.

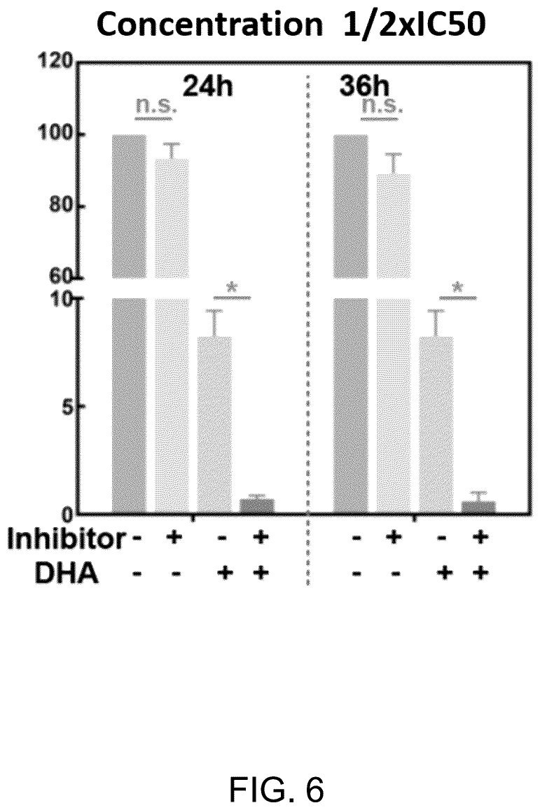

The results are shown in FIG. 6, where the triazoloquinoline derivative was represented by inhibitor; “−” represents the absence of the corresponding substance, and “+” represents the presence of the corresponding substance. The results show that the combination of the triazoloquinoline derivative with DHA can more effectively kill artemisinin-resistant Plasmodium falciparum strains, suggesting that the small molecule can be used as a combination drug to mitigate the spread of artemisinin resistance.

Obviously, the above examples of the present invention are only instances for clearly illustrating the present invention, rather than limiting the embodiments of the present invention. For those of ordinary skill in the art, other variations and modifications in different forms may also be made based on the above description. It is neither necessary nor feasible to exhaustively list all embodiments here. Any amendment, equivalent replacement, improvement, etc., made within the spirit and principle of the present invention shall fall within the scope of protection of the claims of the present invention.

What is claimed is:

1. A transcription factor PfAP2-O5 inhibitor for Plasmodium falciparum, taking a triazoloquinoline derivative comprising a structure represented by formula (I) or a pharmaceutically acceptable salt or hydrate thereof as an active ingredient, and a pharmaceutically acceptable carrier, diluent, or excipient as an adjuvant;

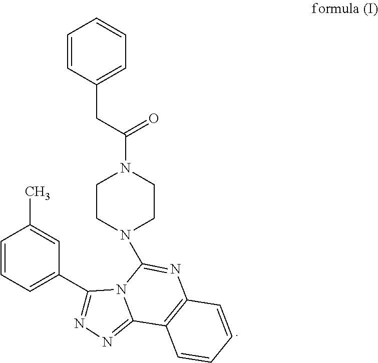 [SMILES: Cc1cccc(-c2nnc3c4ccccc4nc(N4CCN(C(=O)Cc5ccccc5)CC4)n23)c1]

2. A pharmaceutical composition, taking a triazoloquinoline derivative comprising a structure represented by formula (I) or a pharmaceutically acceptable salt or hydrate thereof as an active ingredient, and a pharmaceutically acceptable carrier, diluent, or excipient as an adjuvant;

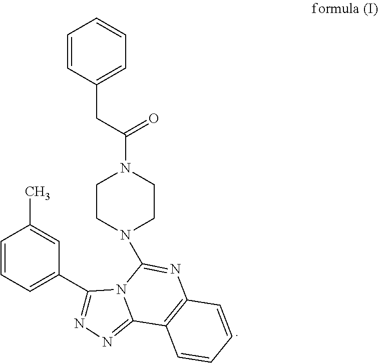 [SMILES: Cc1cccc(-c2nnc3c4ccccc4nc(N4CCN(C(=O)Cc5ccccc5)CC4)n23)c1]

3. The pharmaceutical composition according to claim 2, wherein the active ingredient further comprises an artemisinin-based compound.

4. The pharmaceutical composition according to claim 3, wherein the artemisinin-based compound is a dihydroartemisinin-based compound.

5. The pharmaceutical composition according to claim 3, wherein in the pharmaceutical composition, a molar ratio of the triazoloquinoline derivative of the structure represented by formula (I) or the pharmaceutically acceptable salt or hydrate thereof to the artemisinin-based compound is 1:500 to 500:1.

6. The pharmaceutical composition according to claim 2, wherein the pharmaceutical composition is prepared in an oral dosage form or an injection dosage form.

7. A drug for treating malaria resistant to artemisinin and an analog thereof, taking a triazoloquinoline derivative comprising a structure represented by formula (I) or a pharmaceutically acceptable salt or hydrate thereof as an active ingredient, and a pharmaceutically acceptable carrier, diluent, or excipient as an adjuvant; 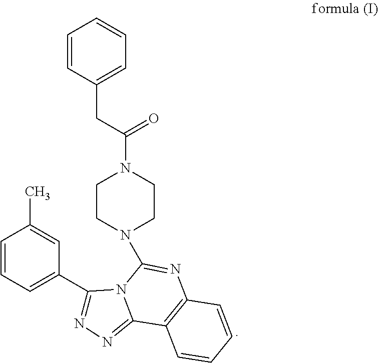 [SMILES: Cc1cccc(-c2nnc3c4ccccc4nc(N4CCN(C(=O)Cc5ccccc5)CC4)n23)c1]
